(19) United States
(12) Patent Application Publication
(10) Pub. No.: US 2020/0170669 A1
(43) Pub. Date: Jun. 4, 2020

(54) ENDOVASCULAR SHEATH AND RELATED METHODS

(71) Applicant: Mark C. GOLDBERG, Beachwood, OH (US)

(72) Inventor: Mark C. GOLDBERG, Beachwood, OH (US)

(21) Appl. No.: 16/250,030

(22) Filed: Jan. 17, 2019

Related U.S. Application Data
(63) Continuation-in-part of application No. 16/209,133, filed on Dec. 4, 2018.

(51) Int. Cl. A61B 17/34; A61B 90/11

(52) U.S. Cl. CPC A61B 17/3423; A61B 2017/00778; A61B 2017/3425; A61B 90/11

(57) ABSTRACT

Various embodiments of an endovascular sheath include a shaft portion with an interior lumen and an exit opening. The sheath may be introduced into a blood vessel along a guidewire that has already been placed within the vessel (typically via a Seldinger needle technique). Along the shaft is an opening that permits exit of a second guidewire introduced into the sheath lumen. The sheath may include, within the lumen, one or more features that encourage exit of the second guidewire through the opening at an optimal angle. A secondary lumen carries a stiffening wire for procedures involving tortuous vascular anatomy.

### RELATED APPLICATION

This is a continuation-in-part of U.S. Ser. No. 16/209,133, filed on Dec. 4, 2018, the entire disclosure of which is hereby incorporated by reference.

### TECHNICAL FIELD

The present invention relates generally to vascular treatment procedures, and in particular to devices for providing vascular access and directional control of a guidewire.

### BACKGROUND

Endovascular procedures involve surgical access to venous or arterial blood vessels. These procedures can be surgical (e.g., relief of occlusion), therapeutic (e.g., involving the intravascular administration of fluids or medications such as analgesics), or diagnostic (e.g., monitoring of intravascular parameters such as arterial or venous pressure).

An endovascular procedure entails obtaining access into the vasculature and maintaining it for the duration of the procedure. This is most commonly done by placing an “introducer” endovascular sheath in the blood vessel to enable passage of the interventional instruments in and out without losing the entry point or causing damage to the vessel. Placement of an endovascular sheath may be performed using the modified Seldinger technique. This involves puncture of the vessel with a needle, passage of a guidewire through the needle, removal of the needle, incision of the skin, placement of a catheter sheath over the guide-wire, and ultimately, removal of the guide-wire and any interventional instruments that were introduced.

A “retrograde” puncture is against the direction of blood flow from the heart, and “antegrade” puncture is in line with the direction of blood flow. For example, a commonly accessed vessel is the common femoral artery (CFA). If the access sheath is directed toward the aorta and away from the leg, it is termed retrograde access. Conversely if the sheath is directed toward the patient's foot, and in the direction of blood flow, it is termed antegrade.

Some procedures involve access to more than one vessel. For example, the CFA bifurcates into the profunda femoris (PF) and the right superficial femoral artery (SFA), and it may be necessary to access both vessels. Due to anatomical constraints, accessing either the PF or the SFA generally occurs through a puncture in the CFA, which can be reached through the skin; the guidewire is then sent into the vessel of interest. Because of the bifurcation, however, it can be difficult to ensure entry of a guidewire into the correct vessel. Suppose, for example, a patient has a blockage 102 in his SFA as shown in FIG. 1. A vascular interventionist accesses the CFA below the inguinal ligament but above the bifurcation, and threads a guidewire 105 with a cutting tip into the SFA, necessitating a bend in the region indicated at 107. If the bend is too severe or the occlusion is at the origin of the SFA, the guidewire may enter the PF rather than the SFA. Manipulating the guidewire 105 to follow the proper route toward the blockage 105 without causing injury to the vessels can be challenging. Moreover, using conventional techniques, it can be difficult to achieve stable placement of a catheter sheath at a bifurcation even after a guidewire has been properly advanced into the vessel of interest.

When the anatomy of branch vessels becomes tortuous and serpentine, advancement of wires and devices can be even more challenging, as the shape of the vessels can cause retraction and telescoping of the guidewire in the opposite direction. The aortic arch—the portion of the main artery that bends between the ascending and descending aorta—is representative of such vessels and must be traversed in carotid stenting procedures. Indeed, most technical failures in carotid stenting are related to a complex aortic arch, and this has led to a classification system (aortic arch elongation classification) reflecting various levels of procedural difficulty in vessel cannulation. This is illustrated in FIG. 1, with CCA referring to the common carotid artery. While guidewire flexibility is essential, the vessel curvatures mandating increased flexibility are precisely the ones that promote failure modes such as retraction and telescoping, and guidewire flexibility only exacerbates these vulnerabilities.

Accordingly, there is a need for improved means facilitating access to vasculature, particularly tortuous and serpentine vessels.

### SUMMARY

Various embodiments of an endovascular sheath in accordance herewith include a shaft portion with primary and secondary lumens, and an exit opening. The sheath may be introduced into a blood vessel along a guidewire that has already been placed within the vessel (typically via a Seldinger needle technique). Along the shaft is an opening that permits exit of a second guidewire introduced into the primary sheath lumen. A third guidewire is passed through the secondary lumen to stiffen the device. The sheath may include, within the primary lumen, one or more features that encourage exit of the second guidewire through the opening at an optimal angle. For example, the primary lumen may include a ramp or other internal feature angled to direct exit of the second guidewire at approximately the appropriate angle to pass into an adjoining vessel. Following advancement of the second guidewire, a second sheath or catheter may be passed therealong through the device, also exiting via the opening. This second sheath branches off, e.g., into another vessel that would be difficult to access with a single sheath due to the tortuous path involved.

Accordingly, the implementations described herein may provide reliable placement of multiple intravascular guidewires and sheaths/catheters notwithstanding extreme vessel anatomies. The device may be used at branch vessel bifurcations or for directional control within the main trunk of a blood vessel prior to a bifurcation, e.g., to re-direct and change the course of the wire up to 180°. This permits the guide wire to be placed in a 180° orientation to the original placement of the sheath, and if the sheath is removed, the guidewire will now permit introduction of a second sheath along the newly established course change. Thus if the original sheath and guidewire is oriented in an antegrade position, then using this technique, the second sheath can be re-directed in a retrograde fashion using the original access point without an additional puncture/access of the vessel.

Therefore, in a first aspect, the invention pertains to an endovascular introducer sheath. In various embodiments, the introducer sheath comprises an elongated body comprising a head portion and, extending therefrom, a shaft portion including primary and secondary lumens, wherein (i) the primary lumen terminates in an open distal end, (ii) the primary lumen is sized to accommodate a plurality of guidewires, and (iii) the secondary lumen extends parallel to but is physically isolated from the primary lumen and is sized to receive a stiffening wire; an opening through the shaft portion to the primary lumen but not the secondary lumen, the opening being spaced apart from the distal end; and means for directing a guidewire received at the head portion out the opening at an exit angle.

In some embodiments, the exit angle is no greater than 90° relative to the shaft portion. The directing means may be a ramp within the interior lumen; the ramp may descend into the primary lumen from a distal peripheral edge of the opening. In some embodiments, the ramp has a scooped profile and extends into the lumen sufficiently to cause contact between a lower edge of the ramp and a guidewire positioned against the interior lumen opposite the opening.

The introducer sheath may include an inflatable member within the primary lumen and an inflation channel fluidically coupled to the inflatable member, where at least a portion of the inflatable member is opposed to the opening. In some embodiments, a portion of the inflatable member may be directly opposed to the opening. The inflatable member may reside on an exterior surface of the shaft and substantially surrounding but not occluding the opening, e.g., having the form of a cuff.

The opening is generally sufficiently large to allow therethrough a sheath advanced along the second guidewire. In some embodiments, the distal end has a pigtail configuration.

In another aspect, the invention relates to a method of accessing a branched blood vessel along a tortuous endovascular path comprising an arch and a target vessel branching therefrom. In various embodiments, the method comprises the steps of introducing a first sheath into the arch by inserting it into a remote extracorporeal site and guiding the sheath over a first guidewire, where the first sheath comprises (a) an elongated body comprising a head portion and, extending therefrom, a shaft portion including primary and secondary lumens, wherein (i) the primary lumen terminates in an open distal end, (ii) the primary lumen is sized to accommodate a plurality of guidewires, and (iii) the secondary lumen extends parallel to but is physically isolated from the primary lumen; and (b) an opening through the shaft portion to the primary lumen but not the secondary lumen, the opening being spaced apart from the distal end; positioning the opening opposite the target vessel; directing a second guidewire received at the head portion out the opening at an exit angle, the exit angle conforming to an angle of the target vessel relative to the shaft portion; and directing a second sheath into the target vessel along the second guidewire.

The method may further comprise the step of stabilizing the second sheath following introduction thereof into the target vessel using an inflatable member. For example, the inflatable member may disposed within the primary lumen with at least a portion of the inflatable member being opposed to the opening; or the inflatable member may be disposed on an exterior surface of the shaft, substantially surrounding but not occluding the opening. In the latter case, the inflatable member may have the form of a cuff surrounding the opening.

In some embodiments, the method further comprises the step of advancing a stiffening wire through the first sheath before advancing the second sheath therethrough. The first and second guidewires and the stiffening wire may be left in place following placement of the second sheath in the target vessel.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 illustrates the anatomy of a representative placement region for the endovascular devices described herein.

FIG. 2 is a plan view of an endovascular sheath including a mid-shaft opening in accordance with an embodiment of the present invention.

FIG. 3A is a partial sectional view illustrating introduction into a blood vessel of the sheath shown in FIG. 2.

FIG. 3B shows a sheath having a curvature.

FIG. 4A is a schematic section illustrating the sheath shown in FIG. 2 extending into the PF, and FIG. 4B shows an emerging guidewire (or shaped catheter in combination with wire) oriented toward the SFA.

FIGS. 5A and 5B are schematic sections of a portion of the sheath as shown in the previous drawings, illustrating a ramp feature.

FIG. 6 is a sectional schematic illustrating an alternative embodiment of the invention in which the end of the shaft distal to the opening has tapered profile.

FIG. 7A is a perspective view of an embodiment of the invention having a slit, and FIG. 7B illustrates an embodiment having the ramp feature shown in FIG. 5 with a groove therein and running along the terminal length of the sheath.

FIG. 8 is a schematic perspective view of an alternative embodiment of the present invention including an elongated slit opening with tapered distal shaft.

FIG. 9 schematically illustrates use of an embodiment of the invention in a tortuous vascular environment.

FIG. 10 is a schematic section illustrating an embodiment of the invention having a secondary lumen for receiving a stiffening wire.

FIG. 11A is a cross-section taken at the line 11A-11A in FIG. 10, illustrating an embodiment with the secondary lumen in the interior of the device.

FIG. 11B is a cross-section of an alternative embodiment in which the secondary lumen extends along the exterior of the device.

FIG. 12 is a sectional view of an embodiment with an interior retention balloon.

FIGS. 13A and 13B are plan and perspective views, respectively, of an embodiment with an exterior cuff balloon.

FIG. 14 schematically illustrates use of various embodiments hereof in tortuous vascular environments.

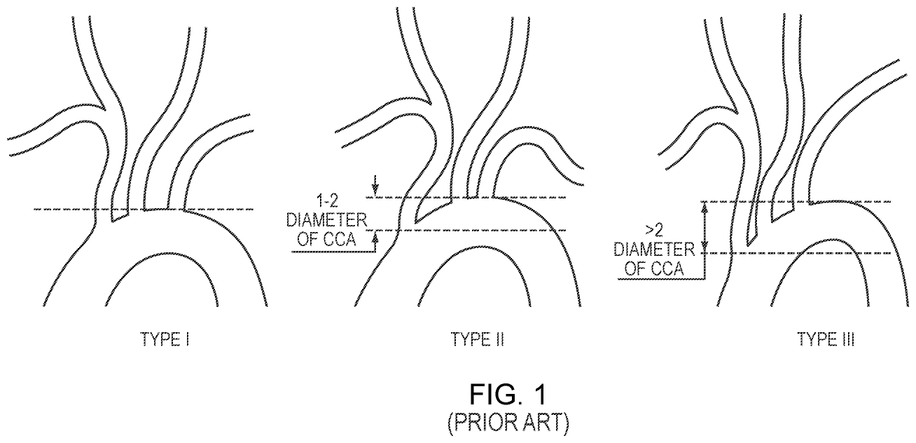

### DETAILED DESCRIPTION

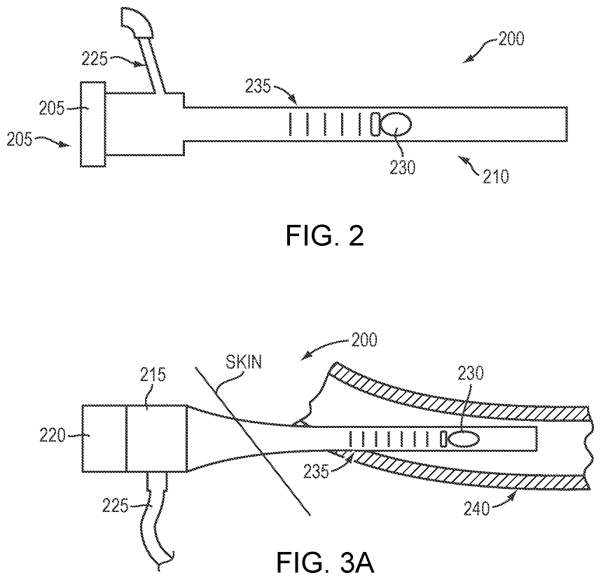

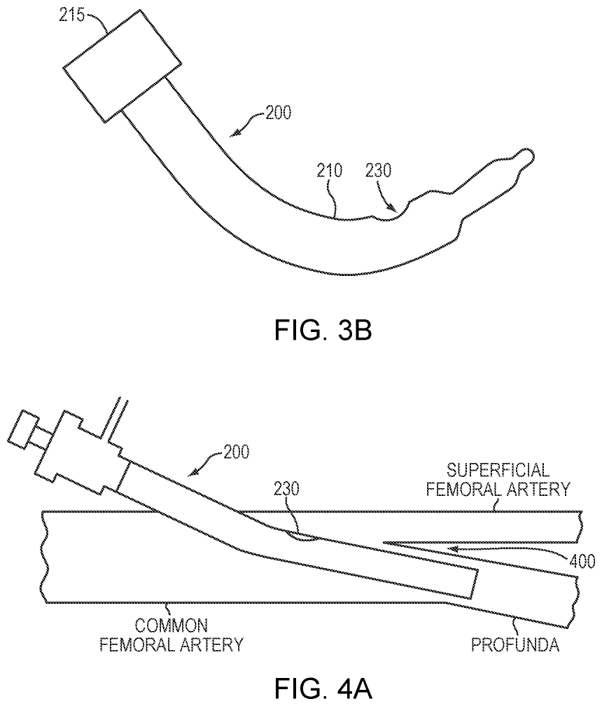

Refer first to FIGS. 2 and 3A, which illustrate an endovascular sheath 200 in accordance with embodiments of the invention and its use. The sheath 200 includes a head portion 205 and a shaft portion 210. The head portion 205 is conventional and includes a receptacle 215 for receiving a dilator instrument 220, which is shown inserted into the sheath 200 in FIG. 3A. The head portion 205 also includes a tube and valve assembly 225 for irrigation or administration of medication during a procedure. The shaft portion 210 includes an opening 230, described in greater detail below, and a series of radiopaque (e.g., platinum/iridium) marker bands 235 to enable visibility of the sheath 100 under fluoroscopy. The shaft portion may be straight or, as illustrated in FIG. 3B, may have a curvature ranging from 0 to 40%. If the shaft portion 210 is curved, the opening 230 may be located on the inner curvature or the outer curvature.

The sheath 200 may be made from metal or any suitable polymeric material such as plastic (e.g., PTFE, FEP, PFA, PE, one or more polyamides, one or more polyimides, a thermoplastic elastomer such as PEBAX, or one or more urethane polymers), and may be provided with a hydrophilc coating to minimize vessel trauma.

During a procedure, a guidewire is first inserted via a needle into the blood vessel 240 to which access is desired. A wire is advanced through the needle and then, maintaining the wire, the needle is removed. The sheath 200 is introduced through the skin and into the vessel 240 along the guidewire. If used, the dilator 220 is first inserted through the head portion 215 of the sheath 200 and the tapered distal end thereof (not shown) emerges from the distal end of the sheath 200. The tapered dilator eases the passage of the sheath 200 into the vessel 240 and is removed following placement.

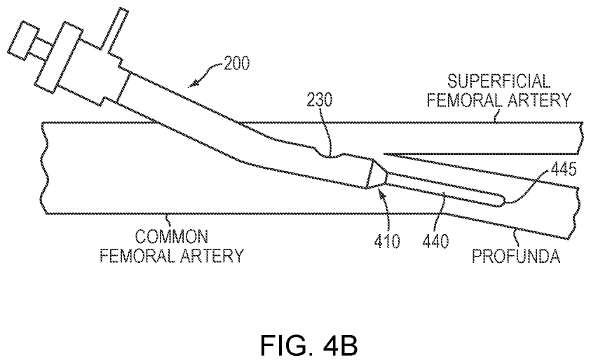

With reference to FIGS. 4A and 4B, the sheath 200 facilitates convenient and reliable access to one of two blood vessels at a bifurcation 400. During a procedure involving the PF and the SFA, as illustrated, the guidewire is extended into the PF and the sheath 200 is thereupon inserted along the guidewire into the PF. The distal end of the sheath 200 may be positioned in the PF so as to remain anchored therein as a second guidewire is introduced into the sheath 200. For example, a balloon at the distal end of the sheath may be inflated to retain the sheath in the desired position and serves to provide a stable platform or structural support for the control and advancement of the guidewire. As detailed below, the sheath 200 is configured such that the second guidewire emerges from the window 230, preferably oriented at an angle that will lead it into the desired vessel branch (the SFA in FIG. 4B). This can be accomplished in various ways. One way exploits a curvature in the sheath, up to 40%, which may be rotated using the PF as a fulcrum, and thus used to optimize the orientation of the window 230 relative to the SFA. Another way is utilizing a shaped catheter through the window in combination with the curvature of the sheath. By rotation of the sheath and rotation of the shaped catheter, precise alignment with the target branch vessel can be achieved.

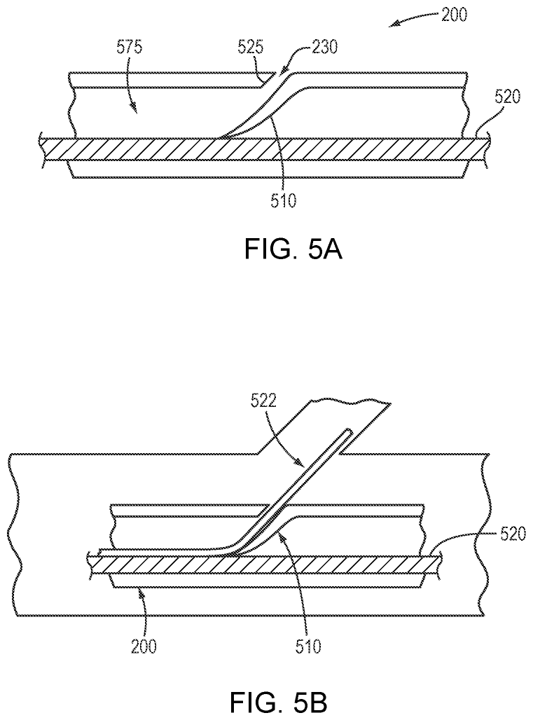

As illustrated in FIGS. 5A and 5B, the sheath 200 may include a ramp structure descending from the window 230 into the lumen 510 of the sheath 200. The ramp structure may have a scooped profile terminating in a fine lower edge, and extend 180° or less around the distal peripheral edge of the window 230. In this way, when the sheath 200 is inserted into a vessel along a guidewire 520, the lower edge of the ramp 210 rides along the guidewire surface. When a second guidewire 522 is advanced through the lumen 515 (which may occur before or after removal of the first guidewire), it travels above the first guidewire 520 and, when it encounters the ramp 510, follows the ramp and exits through the window 230. The fine edge of the ramp 510 ensures that the second guidewire does not meet resistance when it strikes the edge. The angle of travel of the guidewire as it exits the window 230 may be dictated by a bevel around the peripheral edge of the window 230. For example, the window 230 may be sized to be only slightly larger than the second guidewire; the proximal edge 525 of the window 230 may have a bevel angle matching that of the ramp 510 as it meets the distal portion of the window 230, and the second guidewire emerges from the window 230 at the angle of these surfaces. In this way, if desired, the sheath 200 can be configured for particular procedures where the angle of the bifurcation 400 is known—for example, given the anatomy of the average patient, the bevel angle can be selected to ensure, with the sheath 200 inserted into one bifurcation to a specified one of the marking bands 235, that the path of the emerging second guidewire will properly enter the second vessel bifurcation. Furthermore, the lumen 515 may be shaped (i.e., have a cross-sectional profile) to enforce travel of the second guidewire above the first; for example, the lumen 515 may be off-round, e.g., ovoid or elliptical in cross-section.

Alternatively, the lumen 515 may be sized to accommodate a dilator (which rides along the first guidewire during insertion) as well as a second guidewire, which once again emerges from the window 230 as described above. Once the sheath 200 has been introduced and the second guidewire advanced into the bifurcated vessel, the dilator is removed.

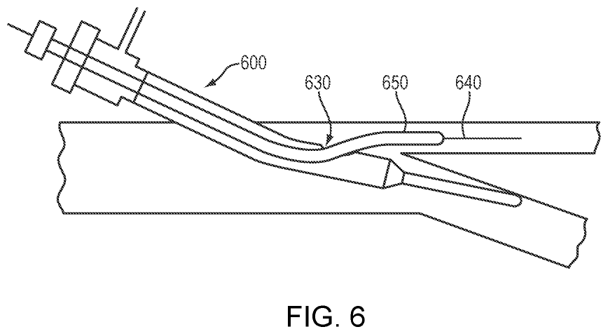

In an alternative approach, the interior or exterior surface of the sheath 200 may be tapered, and it is the taper (more specifically, the effect of the taper on the diameter of the interior lumen) that forces the second guidewire out the window 230, which, once again, may be beveled to enforce a desired exit angle. One example of this approach is illustrated in FIGS. 4B and 6, the latter showing a sheath 600 with a head portion and a shaft portion, and a window 630 through the shaft portion. The shaft portion includes a tapered distal end portion 440. The terminus 445 of the distal end may be generally conical or bullet-shaped to facilitate smooth travel through the skin and vessel wall, with an opening through which the first guidewire passes during placement. This configuration avoids the need for a dilator. The lumen diameter in the tapered portion may be just large enough for a guidewire (e.g., 0.025 inch or 0.64 mm) to slidably pass through, and the taper begins just distal of the window 230/630 as indicated at 410. An additional wire 640 advanced into the sheath 600 encounters the smaller cross-sectional lumen area of the taper and is thereby forced through the window 630, which may be shaped, as noted above, to enforce a desired exit directionality. In effect, the constricting lumen diameter serves as a “ramp” to the window 630. In addition to avoiding use of a dilator, this approach also minimizes blood communication between the distal shaft end and the lumen of the sheath 600 given the close fit to the guidewire 640. A secondary sheath 650 may be inserted along the guidewire 640 and out the exit window 630, as illustrated.

As noted, the exit angle depends on, and may be close or equal to, the angle of bifurcation between the two vessels with which the device is to be used. In general, the exit angle—that is, the angle between the guidewire as it emerges from the window and the exterior surface of the sheath—is less than 90°, and it most cases less than 45°. For example, the branching angle between the profunda and the SFA may range, for example, from 30° to 35°.

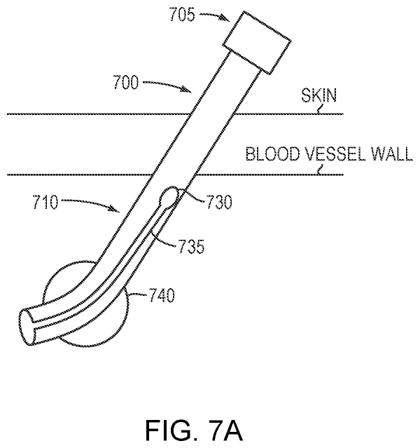

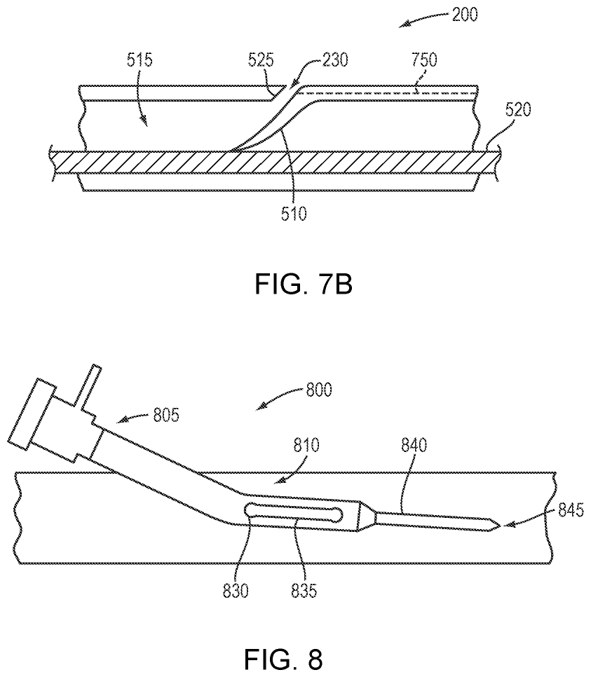

During some procedures, the sheath as described herein may be removed following placement of the second guidewire and, in some cases, replaced with a conventional catheter sheath before, for example, a cutting operation is performed. To ease removal of the sheath with the second guidewire in place, a groove or slit may be included in the shaft portion. Such a slit is illustrated in FIG. 7A, which shows a sheath 700 having a head portion 705 and a shaft portion 710, and constructed as described above. Once again the sheath 700 includes a window 730 for placement of the second guidewire and also a slit 730 through the wall of the shaft portion 710, extending from the window 730 to the terminus of the shaft portion 710. In some embodiments, the window 730 is merely the origin of the slit 735—i.e., the width of the slit 735 is the same as the diameter of the window 730. In other embodiments, the width of the slit 735 is wider or (more typically) narrower than the diameter of the window 730. FIG. 7A also illustrates a conventional balloon associated with the shaft portion 710, and which may be inflated via the head portion 705 to anchor the sheath 700 within a blood vessel.

When the sheath 700 is retracted from a blood vessel, the slit 735 allows the second guidewire to enter the lumen of the sheath 700, thereby avoiding harm to the interior wall of the blood vessel that could occur if, for example, the guidewire were to be forced against the blood-vessel wall by the exterior surface of the sheath 700. A similar protective function can be achieved using a groove or elongated depression in the surface of the shaft portion 710, between window 730 and the terminus of the sheath 700, rather than a slit penetrating all the way through the shaft wall. Indeed, it may not be possible to employ a slit along the full window-to-terminus distance depending on the means used to direct the second guidewire out the window 730. As shown in FIG. 7B, for example, the ramp 510 extends to the surface of the sheath wall and a slit therethrough would defeat its function. However, a groove 750 will not interfere with the ability of the ramp 510 to guide a wire out through the window 230 but, when extended along the shaft to its terminus, will be capable of receiving the wire during retraction of the sheath to thereby prevent compression of the wire against the inner wall of the blood vessel. If desired, the groove 750 can become a slit at a point sufficiently distal to the ramp 510 so as not to affect its mechanical integrity.

FIG. 8 illustrates a variation in which a slit is used with the embodiment having a tapered shaft portion. In particular, the sheath 800 includes a head portion 805 and a shaft portion 810, and a window 830 through the shaft portion. The window 830 extends into a slit 835. The terminus 845 of the distal end 840 may be generally conical or bullet-shaped to facilitate smooth travel through the skin and vessel wall, with an opening through which the first guidewire passes during placement. The lumen diameter in the tapered portion 640 may be just large enough for a guidewire to slidably pass through, and the taper begins just distal of the end of the slit 835. In this configuration, it is unnecessary for the slit 835 (or a groove) to extend to the terminus 845 of the shaft portion because the diminishing diameter of the shaft distal to the end of the slit 835 already prevents or reduces the likelihood of contact between the guidewire and the inner wall of the blood vessel.

An advantage of the invention is its ability to provide support and stability in a complex vascular environment. When a sheath in accordance with the invention is placed into the PF or another vessel, contact between the skin and the end of the sheath helps secure and reinforce the shaft portion. When an instrument such as a wire or catheter is then advanced through the mid-shaft opening, it is mechanically supported by the sheath. In conventional procedures, the force applied to a wire or catheter and transferred by contact to a calcified occlusion without support often results in buckling and redirection of the instrument (e.g., into a branching vessel such as the PF). The support and stability provided by a sheath in accordance herewith, by contrast, helps prevent the unwanted redirection of the wire and thereby sustains application of force at the target.

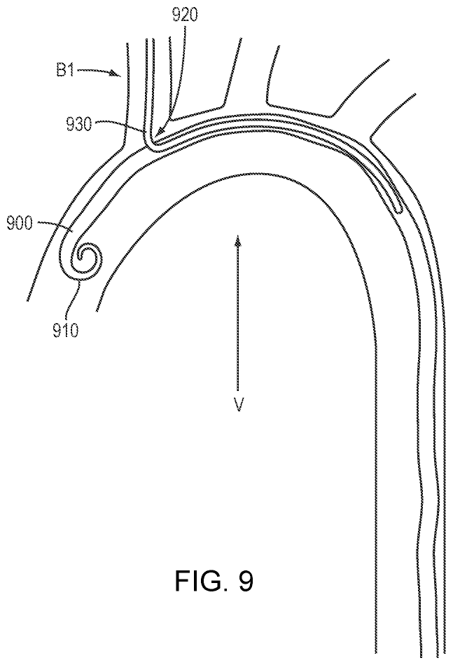

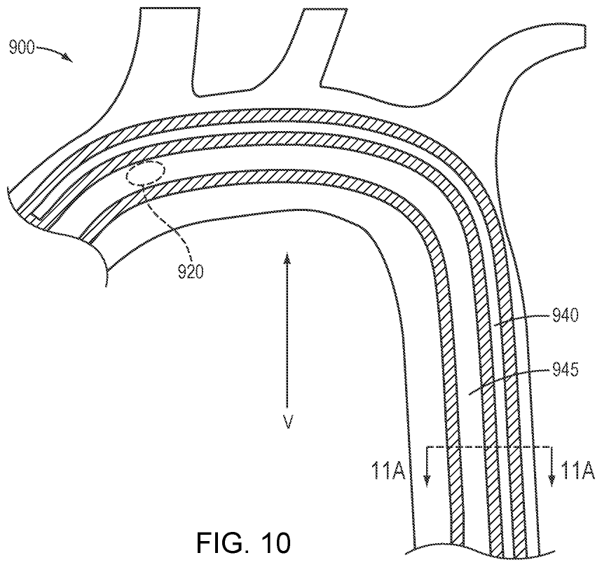

When the anatomy of branch vessels becomes tortuous and serpentine, advancement of wires and devices can cause retraction and telescoping in the opposite direction. As illustrated in FIG. 9, a vessel V includes three branches, one of which is labeled B1. A primary sheath 900 terminates in a flexible pigtail end 910 and includes an opening 920 as described above. The sheath 900 may be straightforwardly introduced so as to traverse the curve of the vessel V, but it would be more difficult—and risk retraction and telescoping—if an attempt were made to maneuver it into the branch vessel B1. As a consequence, in accordance herewith, the opening 920 is positioned opposite the branch vessel B1. A guidewire is advanced out the opening 920 and into the vessel B1, and a second sheath 930 is advanced through the original sheath 900 along the guidewire. Upon removal of the guidewire, the clinician has access to the branch vessel B1 via the sheath 930. In a particular example, the vessel V is the ascending aorta and the mid-shaft opening 920 is aligned with the origin of the innominate artery; a catheter or instrument may be advanced all the way into the carotid artery and the sheath 930 will serve as a stable platform. Reducing the risk of recoil or slippage of the instrument out of the carotid and toward the aorta is an important benefit, as it reduces the risk of stroke.

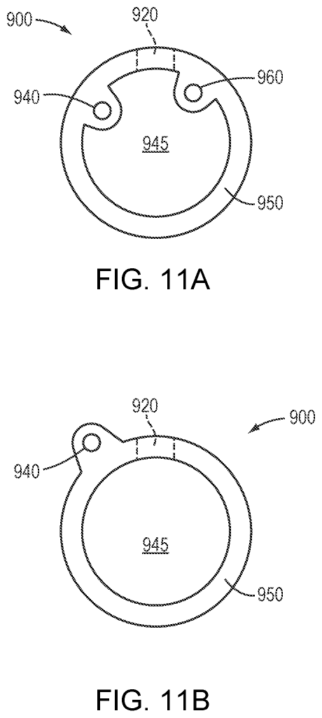

Depending on the anatomy and the nature of the arch elongation, the type of arch and branch variants, it may be desirable to stiffen the sheath 900 as illustrated in FIGS. 10, 11A, and 11B. With reference to FIG. 10, the sheath 900 includes an additional passage or lumen 940 through which a stiffening guidewire (e.g., 0.038 inch) may be introduced. The lumen 940 may terminate within the shaft of the sheath 900, i.e., not extend to the distal end of the device. The additional guidewire confers stability to the sheath 900 and discourages flexing and retraction notwithstanding the curvature of the arched vessel V.

The lumen 940 is physically isolated from (i.e., does not communicate with) the primary lumen 945. FIGS. 11A and 11B illustrate alternative constructions. In FIG. 11A, the secondary lumen 940 is formed within a rib extending radially into the lumen 945, although if the wall 950 has sufficient thickness, the secondary lumen 940 can be formed within the wall. Also as shown in FIG. 11A, the sheath 900 may include an additional channel 960 to facilitate inflation of an exterior or interior balloon, as described below. FIG. 11B shows the secondary lumen 940 carried in an exterior rib. In general, the interior or in-wall secondary lumen shown in FIG. 11A is preferred, as a uniform, smooth exterior surface offers optimal maneuverability of the sheath through blood vessels, particularly over long anatomic distances. However, the interior rib requires that the primary lumen 945 have a sufficient diameter to accommodate the secondary sheath as well as the rib.

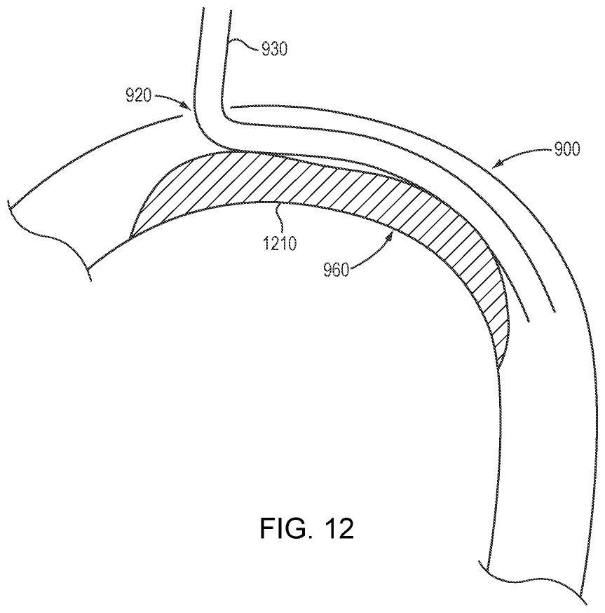

In various embodiments, an inflatable balloon is positioned within or outside the first sheath 900 to retain the second sheath 930 in place when deployed through the window 920 and into the target branch vessel. This precludes movement or retraction of the second sheath 930 following placement. One balloon configuration is shown in FIG. 12. The balloon 1210 is mounted along the interior wall of the primary sheath 900 opposite the window 920 and extending a short distance distal to the window and, preferably, a longer distance proximal to the window. For example, the proximal extension of the balloon relative to the window may be three to five times longer than the distal extension. In embodiments using a ramp to guide the secondary sheath 930 through the window 920, the balloon 1210 may terminate just proximal to the ramp.

The balloon 1210 extends along the air channel 960 described above, which terminates and opens into the balloon 1210. The other end of the air channel 960, at the proximal end of the primary sheath 900, is connected to an inflation device, such as a squeeze bulb, syringe or pump operated by the clinician. Some inflation devices provide controlled measures of pressure based on atmospheric pressure. Suitable inflation devices include the basixTAU device marketed by Merit Medical, South Jordan, Utah. In its uninflated state, the balloon 1210 occupies little volume within the lumen of the primary sheath 900 and does not interfere with travel of the secondary sheath 930 through the primary sheath 900 and out the window 920; when inflated, as illustrated in FIG. 12, the balloon 1210 securely retains the deployed secondary sheath and prevents retraction or rotation thereof.

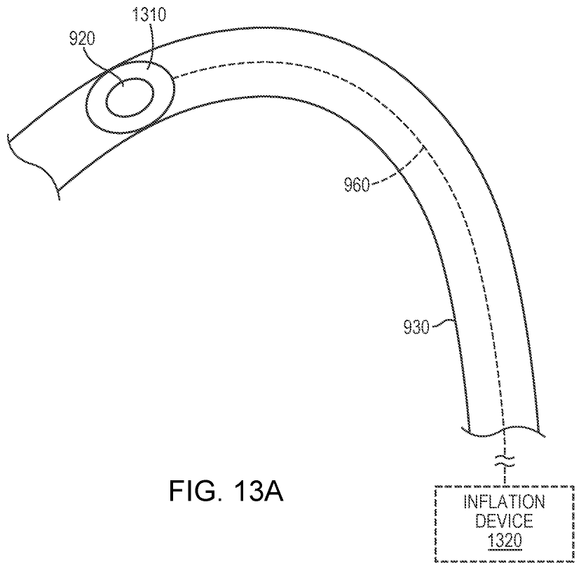

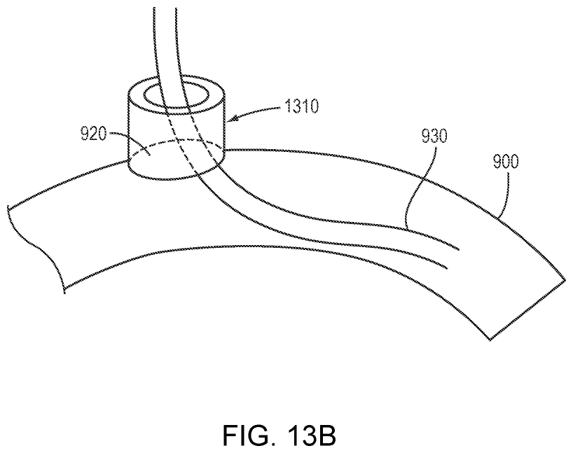

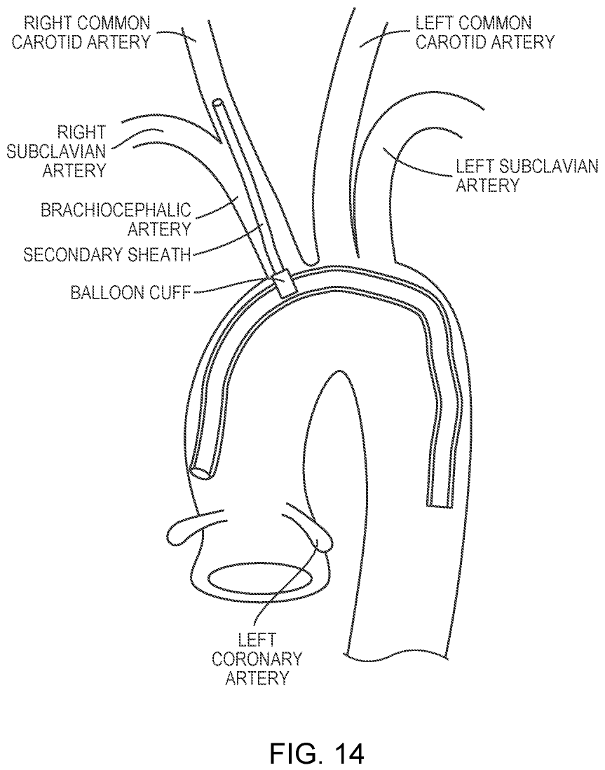

FIGS. 13A and 13B illustrate an alternative embodiment in which the balloon 1310 takes the form of an exterior cuff surrounding the window 920 and, once again, inflated via the air channel 960 and an inflation device 1320. In its uninflated state, the balloon 1310 rests largely flat against the exterior surface of the primary sheath 900 and does not interfere with the passage of the sheath 900 through vasculature during placement. The balloon 1310 may, for example, be glued on its lower surface with, e.g., medical-grade epoxy to the sheath 920 with an inflation port positioned against the terminal opening of the air channel 960. The inflation port is sealed against air leakaged when the balloon 1310 is glued into place. (An analogous procedure can be used to anchor the interior balloon 1210.) When inflated, as shown in FIG. 13B, the balloon 1310 assumes a toroidal or cylindrical conformation that grips the secondary sheath 930, retaining it relative to the primary sheath 900 and frictionally preventing retraction of the secondary sheath.

It should be noted that depending on the configuration and procedure for which the device is employed, the ramp structure 510 may itself impart sufficient support to the exiting second sheath 930 to prevent movement or retraction without the need for a balloon.

The overall operation of the device in a representative procedure is shown in FIG. 14. A traditional Seldinger technique is used to advance a hollow needle into the common femoral artery in a retrograde fashion, and a wire is advanced through the needle. The needle is withdrawn over the wire and then a conventional sheath is advanced over the wire. A second wire is then advanced into the aortic arch. Optionally at this point, a pigtail catheter can be advanced into the aortic arch and an aortic injection performed to evaluate the anatomy. Maintaining pressure on the insertion site, the conventional sheath is removed and a primary sheath in accordance herewith is advanced into the femoral artery and further advanced under fluoroscopy into the aortic arch. Depending on the arch anatomy and variants of branch vessels, a plan for the intended intervention is determined and the primary sheath may be left in the aortic arch; optionally it may instead be advanced over a wire into the right subclavian artery (for interventions on the right carotid and not on the left carotid). Depending on the anatomy and the nature of the arch elongation, as well as the type of arch and branch variants, the option for stiffening the sheath may be necessary. A stiff wire can be advanced through an orifice external to the patient, near or within the hub of the primary sheath, through the secondary lumen. The wire makes the sheath into a stable platform that is unlikely to retract or flex. The window or slit is aligned with the origin of the aortic branch vessel and a catheter and wire are advanced into this branch vessel. Now with a wire in place, the catheter is removed and a new guide catheter/sheath is advanced over the wire in the branch vessel. If desirable to create stability, a balloon is inflated to secure the guide catheter to the outer platform sheath. At this point the remaining steps of the vascular procedure are carried out.

Thus, in various embodiments, support within complex branch vessels is provided by the primary sheath (which may, for example, anchor in the aorta); the window or opening aligned with the target branch vessel, and the secondary sheath when advanced into the target vessel and immobilized using the inflatable member. Up to three guidewires can be used simultaneously: the stiff wire over which the primary sheath is initially advanced, and which may remain in place; the stiffening wire advanced through the secondary lumen; and the third guidewire over which the secondary sheath is advanced through the mid-shaft opening.

It is to be understood that the features of the various embodiments described herein are not necessarily mutually exclusive and can exist in various combinations and permutations, even if such combinations or permutations are not made express herein, without departing from the spirit and scope of the invention. In fact, variations, modifications, and other implementations of what is described herein will occur to those of ordinary skill in the art without departing from the spirit and the scope of the invention.

What is claimed is:

1. An endovascular introducer sheath comprising:
a. an elongated body comprising a head portion and, extending therefrom, a shaft portion including primary and secondary lumens, wherein:
i) the primary lumen terminates in an open distal end;
ii) the primary lumen is sized to accommodate a plurality of guidewires; and
iii) the secondary lumen extends parallel to but is physically isolated from the primary lumen and is sized to receive a stiffening wire;
b. an opening through the shaft portion to the primary lumen but not the secondary lumen, the opening being spaced apart from the distal end; and
c. means for directing a guidewire received at the head portion out the opening at an exit angle.

2. The introducer sheath of claim 1, wherein the exit angle is no greater than 90° relative to the shaft portion.

3. The introducer sheath of claim 1, wherein the directing means is a ramp within the interior lumen.

4. The introducer sheath of claim 3, wherein the ramp descends into the primary lumen from a distal peripheral edge of the opening.

5. The introducer sheath of claim 4, wherein the ramp has a scooped profile and extends into the lumen sufficiently to cause contact between a lower edge of the ramp and a guidewire positioned against the interior lumen opposite the opening.

6. The introducer sheath of claim 1, further comprising an inflatable member within the primary lumen and an inflation channel fluidically coupled to the inflatable member, at least a portion of the inflatable member being opposed to the opening.

7. The introducer sheath of claim 6, wherein a portion of the inflatable member is directly opposed to the opening.

8. The introducer sheath of claim 1, further comprising an inflatable member on an exterior surface of the shaft and substantially surrounding but not occluding the opening, and an inflation channel fluidically coupled to the inflatable member.

9. The introducer sheath of claim 8, wherein the inflatable member has the form of a cuff surrounding the opening.

10. The introducer sheath of claim 1, wherein the distal end has a pigtail configuration.

11. The introducer sheath of claim 1, wherein the opening is sufficiently large to allow therethrough a sheath advanced along the second guidewire.

12. A method of accessing a branched blood vessel along a tortuous endovascular path comprising an arch and a target vessel branching therefrom, the method comprising the steps of:
introducing a first sheath into the arch by inserting it into a remote extracorporeal site and guiding the sheath over a first guidewire, the first sheath comprising (a) an elongated body comprising a head portion and, extending therefrom, a shaft portion including primary and secondary lumens, wherein (i) the primary lumen terminates in an open distal end, (ii) the primary lumen is sized to accommodate a plurality of guidewires, and (iii) the secondary lumen extends parallel to but is physically isolated from the primary lumen; and (b) an opening through the shaft portion to the primary lumen but not the secondary lumen, the opening being spaced apart from the distal end;
positioning the opening opposite the target vessel;
directing a second guidewire received at the head portion out the opening at an exit angle, the exit angle conforming to an angle of the target vessel relative to the shaft portion; and
directing a second sheath into the target vessel along the second guidewire.

13. The method of claim 12, further comprising the step of stabilizing the second sheath following introduction thereof into the target vessel using an inflatable member.

14. The method of claim 13, wherein the inflatable member is disposed within the primary lumen and at least a portion of the inflatable member is opposed to the opening.

15. The method of claim 13, wherein the inflatable member is disposed on an exterior surface of the shaft and substantially surrounds but does not occlude the opening.

16. The method of claim 15, wherein the inflatable member has the form of a cuff surrounding the opening.

17. The method of claim 12, further comprising the step of advancing a stiffening wire through the first sheath before advancing the second sheath therethrough.

18. The method of claim 17, wherein the first and second guidewires and the stiffening wire are left in place following placement of the second sheath in the target vessel.
